(19) United States
(12) Patent Application Publication
(10) Pub. No.: US 2009/0060875 A1
(43) Pub. Date: Mar. 5, 2009

(54) HCV Vaccines for Chronic HCV Patients

(75) Inventors: Jurgen Frisch, Marburg (DE); Erich Tauber, Muchendorf a. d. Donau (AT); Vera Burger, Vienna (AT); Christoph Klade, Neustadt (AT); Michael Buschle, La Chaux-de-Fonds (CH); Katherine Cohen, Vienna (AT)

Correspondence Address: FULBRIGHT & JAWORSKI L.L.P., 600 CONGRESS AVE., SUITE 2400, AUSTIN, TX, 78701, US

(73) Assignee: INTERCELL AG, Vienna (AT)

(21) Appl. No.: 11/577,798

(22) PCT Filed: Sep. 23, 2005
(86) PCT No.: PCT/EP05/54773

§ 371 Date: Apr. 23, 2007

(30) Foreign Application Priority Data
Oct. 29, 2004 (EP) 04450200.3

(51) Int. Cl. A61K 38/21; A61K 39/00; A61P 31/00; A61K 38/00

(52) U.S. Cl. 424/85.7; 424/228.1; 514/13; 514/12

(57) ABSTRACT

The invention discloses the use of polycationic compounds for the preparation and manufacturing of medicaments, pharmaceutical compositions, especially vaccines for the treatment of patients with HCV chronic infections.

This application is a national phase application under 35 U.S.C. § 371 of International Application No. PCT/EP2005/054773 filed 23 Sep. 2005, which claims priority to European Patent Application No. 04450200.3 filed 29 Oct. 2004. The entire text of each of the above-referenced disclosures is specifically incorporated herein by reference without disclaimer.

Hepatitis C Virus (HCV) is a member of the flaviviridiae chronically infecting about 170 million people worldwide. There are at least 6 HCV genotypes and more than 50 subtypes have been described. In America, Europe and Japan genotypes 1, 2 and 3 are most common. The geographic distribution of HCV genotypes varies greatly with genotype la being predominant in the USA and parts of Western Europe, whereas 1b predominates in Southern and Central Europe (Bellentani 2000). HCV is transmitted through the parenteral or percutan route, and replicates in hepatocytes. About 15% of patients experience acute self-limited hepatitis associated with viral clearance and recovery. About 80% of infected persons become chronic carriers. Infection often persists asymptomatically with slow progression for years, however ultimately HCV is a major cause of cirrhosis, end-stage liver disease and liver cancer (Liang 2000). Strength and quality of both HTL and CTL responses determine whether patients recover (spontaneously or as a consequence of therapy) or develop chronic infection (Liang 2000).

Standard therapy of HCV comprises a combination of pegylated interferon-alpha and the antiviral ribavirin (Fried 2002). Virologic responses (defined as the absence of detectable HCV RNA 24 weeks after cessation of therapy) are, depending on the genotype, however, achieved in only about 50% of HCV patients with the standard therapy. Moreover, there are many side effects associated with both interferon (IFN) and ribavirin. IFN (also in its pegylated form) stimulates the immune system in a non-specific manner, which causes substantial side effects including flu-like syndrome, fever, headache, arthralgia, myalgia, depression, weight loss, alopecia, leukopenia and thrombopenia⁶. These side effects can necessitate cessation of treatment for some subjects. Treatment with IFN is also contraindicated in subjects with pre-existing hematologic disease (leukopenia and thrombopenia due to liver cirrhosis with splenomegaly). Ribavirin can cause transient liver enzyme elevations and psychiatric symptoms, hemolysis and anemia and has been associated with myocardial infarction in subjects with coronary heart disease. Ribavirin also exhibits teratogenic, mutagenic and carcinogenic potency. Therefore, contraception is mandatory for fertile male and female subjects treated with ribavirin. Therefore, the low tolerability and the considerable side effects of this therapy clearly necessitate novel therapeutic intervention including therapeutic vaccines and new treatment options for chronic HCV infection are urgently required, due to the limitations and lack of effective treatments (Cornberg 2002).

The problem underlying the present invention is to overcome the limitations of the standard interferon-alpha/ribavirin combination therapy by providing effective medicaments or pharmaceutical compositions especially a vaccine for the treatment of chronic HCV infections, especially for those patients who had not responded to or only partially responded to or had relapsed from primary standard HCV therapy.

Therefore the present invention provides the use of polycationic compounds for the manufacturing of medicaments, pharmaceutical compositions especially vaccines for the treatment of patients with HCV chronic infections. Preferably, the patients with HCV chronic infections are those who had not responded to, partially responded to or had relapsed from primary standard HCV therapy by a combination of pegylated interferon-alpha and the antiviral ribavirin.

A patient is considered to have relapsed when HCV RNA becomes undetectable on the primary standard HCV therapy with Peg-interferon alpha and Ribavirin but is detected again after disclontinuation of the treatment. Persons in whom HCV RNA levels remain stable on treatment are considered non-responders, while those whose HCV RNA levels decline (e.g. by >2 logs), but never become undetectable, are referred to as partial responders.

The polycationic compound(s) to be used according to the present invention may be any polycationic compound, which shows the characteristic effects according to the WO 97/30721. Preferred polycationic compounds are selected from basic polyppetides, organic polycations, basic polyamino acids or mixtures thereof. These polyamino acids should have a chain length of at least 4 amino acid residues (WO 97/30721). Especially preferred are substances like polylysine, polyarginine and polypeptides containing more than 20%, especially more than 50% of basic amino acids in a range of more than 8, especially more than 20, amino acid residues or mixtures thereof. Other preferred polycations and their pharmaceutical compositions are described in WO 97/30721 (e.g. polyethyleneimine) and WO 99/38528. Preferably these polypeptides contain between 20 and 500 amino acid residues, especially between 30 and 200 residues.

These polycationic compounds may be produced chemically or recombinantly or may be derived from natural sources.

Cationic (poly)peptides may also be anti-microbial with properties as reviewed in {Ganz, T., 1999}. These (poly)peptides may be of prokaryotic or animal or plant origin or may be produced chemically or recombinantly (WO 02/13857). Peptides may also belong to the class of defensins (WO 02/13857). Sequences of such peptides can be, for example, found in the Antimicrobial Sequences Database under the following internet address:
http://www.bbcm.univ.trieste.it/˜tossi/pag2.html

Such host defence peptides or defensives are also a preferred form of the polycationic polymer according to the present invention. Generally, a compound allowing as an end product activation (or down-regulation) of the adaptive immune system, preferably mediated by APCs (including dendritic cells) is used as polycationic polymer.

Especially preferred for use as polycationic substances in the present invention are cathelicidin derived antimicrobial peptides or derivatives thereof (International patent application WO 02/13857, incorporated herein by reference), especially antimicrobial peptides derived from mammalian cathelicidin, preferably from human, bovine or mouse.

Polycationic compounds derived from natural sources include HIV-REV or HIV-TAT (derived cationic peptides, antennapedia peptides, chitosan or other derivatives of chitin) or other peptides derived from these peptides or proteins by biochemical or recombinant production. Other preferred polycationic compounds are cathelin or related or derived substances from cathelin. For example, mouse cathelin is a peptide, which has the amino acid sequence NH2-RLAGLLRKGGEKIGEKLKKIGQKIKNFFQKLVPQPE-COOH (SEQ ID NO:1). Related or derived cathelin substances contain the whole or parts of the cathelin sequence with at least 15-20 amino acid residues. Derivations may include the substitution or modification of the natural amino acids by amino acids, which are not among the 20 standard amino acids. Moreover, further cationic residues may be introduced into such cathelin molecules. These cathelin molecules are preferred to be combined with the antigen. These cathelin molecules surprisingly have turned out to be also effective as an adjuvant for an antigen without the addition of further adjuvants. It is therefore possible to use such cathelin molecules as efficient adjuvants in vaccine formulations with or without further immunactivating substances.

Another preferred polycationic substance to be used according to the present invention is a synthetic peptide containing at least 2 KLK-motifs separated by a linker of 3 to 7 hydrophobic amino acids (International patent application WO 02/32451, incorporated herein by reference). In the WO 02/32451 a type 1 inducing adjuvant (Immunizer) that is able to strongly enhance the immune response to a specific co-administered antigen and therefore constitutes a highly effective adjuvant is disclosed. The adjuvant (Immunizer) according to the WO 02/32451 is a peptide comprising a sequence R₁-XZXZ_{N}XZX-R₂, (SEQ ID NOS:2-6, wherein: xzxzzzxzx=SEQ ID NO:2; xzxzzzzxzx=SEQ ID NO:3; xzxzzzzzxzx=SEQ ID NO:4; xzxzzzzzzxzx=SEQ ID NO:5; xzxzzzzzzzxzx=SEQ ID NO:6) whereby N is a whole number between 3 and 7, preferably 5, X is a positively charged natural and/or non-natural amino acid residue, Z is an amino acid residue selected from the group consisting of L, V, I, F and/or W, and R1 and R2 are selected independently one from the other from the group consisting of —H, —NH2, —COCH3, —COH, a peptide with up to 20 amino acid residues or a peptide reactive group or a peptide linker with or without a peptide; X-R2 may also be an amide, ester or thioester of the C-terminal amino acid residue. A specifically preferred peptide is KLKLLLLLKLK (SEQ ID NO:7).

According to the present invention, the polycationic compounds can be used for the manufacturing of any medicaments, pharmaceutical compositions, especially vaccines which can be used for the treatment of chronic HCV infection, particularly for the treatment of those patients who had not responded to, partially responded to or had relapsed from primary standard HCV therapy by a combination of pegylated interferon-alpha and the antiviral ribavirin.

Preferably, the polycationic compounds are used for the manufacturing of medicaments, pharmaceutical compositions, especially vaccines that comprise HCV antigens, HCV hotspot epitopes and HCV epitopes. Most preferably, the HCV epitopes that are described in WO 01/24822 and PCT/EP2004/007540. Specifically preferred HCV epitopes therefore include: one or more, especially two, three, four, five or six epitopes found in the following table:

| | |
|---|---|
| | SEQ |
| | ID |
| EPITOPES | NO |

| | |
|---|---|
| MWNFISGIQYLAGLSTLPGN | 8 |
| HMWNFISGI | 9 |
| NFISGIQYLAGLSTLPGNPA | 10 |
| HMWNFISGIQYLAGLSTLPGNPA | 11 |
| IGLGKVLVDILAGYGAGVAGALVAFK | 12 |
| AAWYELTPAETTVRLR | 13 |
| DYPYRLWHYPCTVNYTIFKI | 14 |
| DYPYRLWHYPCTVNFTIFKI | 15 |
| AYSQQTRGLL | 16 |
| TAYSQQTRGLLG | 17 |
| SMSYTWTGALITP | 18 |
| SPGALVVGVI | 19 |
| LPRRGPRL | 20 |
| GSIGLGKVLVDILAG | 21 |
| IGLGKVLVDILAGYG | 22 |
| LGKVLVDILAGYGAG | 23 |
| LVDILAGYGAGVAGA | 24 |
| DILAGYGAGVAGALV | 25 |
| LAGYGAGVAGALVAF | 26 |
| AGAAWYELTPAETTV | 27 |
| AAWYELTPAETTVRL | 28 |
| TAYSQQTRGLLGCIV | 29 |
| VVCCSMSYTWTGALITPC | 30 |
| VDYPYRLWHYPCTVNFTIFKVRMYVGGVEHRL | 31 |
| DYPYRLWHYPCTVNF | 32 |
| YPYRLWHYPCTVNFT | 33 |
| LWHYPCTVNFTIFKV | 34 |
| TVNFTIFKVRMYVGG | 35 |
| TIFKVRMYVGGVEHR | 36 |
| VDYPYRLWHYPCTVNYTIFKIRMYVGGVEHRL | 37 |
| DYPYRLWHYPCTVNY | 38 |
| LWHYPCTVNYTIFKI | 39 |
| TVNYTIFKTRMYVGG | 40 |
| TIFKIRMYVGGVEHR | 41 |
| RMYVGGVEHRL | 42 |
| KFPGGGQIVGGVYLLPRRGPRLGVRATRK | 43 |
| KFPGGGQIVGGVYLLPRRGPRL | 44 |
| YLLPRRGPRL | 45 |
| GPRLGVRAT | 46 |
| RLGVRATRK | 47 |
| GYKVLVLNPSVAAT | 48 |
| AYAAQGYKVL | 49 |
| AYAAQGYKVLVLNPSVAAT | 50 |
| DLMGYIPAV | 51 |
| GYIPLVGAPL | 52 |
| DLMGYIPLVGAPL | 53 |
| CINGVCWTV | 54 |
| GEVQVVSTATQSFLAT | 55 |
| GEVQVVSTATQSFLATCINGVCWTV | 56 |
| QYLAGLSTL | 57 |
| DYPYRLWHYPCTVNFTIFKV | 58 |
| DYPYRLWHY | 59 |
| TVNYTIFKI | 60 |
| TINYTIFK | 61 |
| TVNFTIFKV | 62 |
| HYPCTVNYTI | 63 |
| HYPCTVNFTI | 64 |
| RMYVGGVEHR | 65 |
| TPAETTVRL | 66 |
| GWRLLAPITAYSQQTRGLLGCIV | 67 |
| GQGWRLLAPITAYSQ | 68 |
| RLLAPITAY | 69 |
| GQGWRLLAPITAYSQQTRGLLGCIV | 70 |
| GQGWRLLAPITAYSQQTRGLLG | 71 |
| AYSQQTRGL | 72 |
| ILAGYGAGV | 73 |
| VAGALVAFK | 74 |
| GYGAGVAGAL | 75 |
| SMSYTWTGAL | 76 |
| SYTWTGALI | 77 |
| FTDNSSPPAVPQTFQV | 78 |
| LEDRDRSELSPLLLSTTEW | 79 |
| LEDRDRSELSPLLLST | 80 |
| RSELSPLLL | 81 |
| ELSPLLLST | 82 |
| DRDRSELSPL | 83 |
| LEDRDRSEL | 84 |
| LEDRDRSEL | 85 |
| YLVAYQATVCARAQAPPPSWD | 86 |
| YLVAYQATV | 87 |
| MSTNPKPQRKTKRNTNR | 88 |
| PQRKTKRNTNR | 89 |
| QRKTKRNTN | 90 |
| RKTKRNTNR | 91 |
| MSTNPKPQR | 92 |
| MSTNPKPQK | 93 |
| LINTNGSWHINRTALNCNDSL | 94 |
| NGSWHINRTALNCNDSL | 95 |
| LINTNGSWHI | 96 |
| RTALNCNDSL | 97 |
| LINTNGSWHINRTALN | 98 |
| SWHINRTALN | 99 |
| TTILGIGTVLDQAET | 100 |
| TTILGIGTV | 101 |
| TILGIGTVL | 102 |
| FDSSVLCECYDAGAAWYE | 103 |
| FDSSVLCECYDAGCA | 104 |
| VLCECYDAGA | 105 |
| VVLCECYDAGAAWYE | 106 |
| ARLIVFPDLGVRVCEKMALY | 107 |
| ARLIVFPDL | 108 |
| RLIVFPDLGV | 109 |
| RVCEKMALY | 110 |
| AFCSAMYVGDLCGSV | 111 |
| GVLFGLAYFSMVGNW | 112 |
| TRVPYFVRAQGLIRA | 113 |
| TTLLFNILGGWVAAQ | 114 |
| LLFNILGGWV | 115 |

or fragments containing the T-cell epitope (at least 7, preferably at least 8, even more preferred at least 9 amino acid residues long). Preferably, the vaccine according to the present invention contains two or more, even more preferred three or more, especially four or more of such epitopes in combination. Preferred vaccines contain 3, 4, 5, 6 or 7 individual epitopes in one preparation (or two preparations stored and reconstituted seperately and applied together).

The medicament used according to the present invention is preferably used for induction of CD4+ Helper-T-cells and CD8+ cytotoxic T-cells. A specifically preferred field of use is the application of the medicament to a special group of patients: the use of the present medicament for replacing or supplementing a HCV standard therapy with interferon alpha and ribavirin, especially in patients where such standard therapy is not effective or not effective anymore.

Preferably, the medicament according to the present invention is used in combination with standard treatment such as interferon treatment. Preferably, it can be used for inducing type I T-cell responses in chronic HCV patients, and/or for inducing similar T-cell responses as seen during/after successful standard therapy, and/or for increasing responder rates and/or reducing relapse rates after standard therapy. In particular, the medicament according to the present invention is used in clinical trials as late add-on to standard therapy; the results confirm the excellent safety of the medicament, which does not exacerbate the side-effects of standard therapy such as leukopenia.

Preferably the medicament according to the present invention is specifically designed for HCV genotype 1. Genotype 1 patients have shown the lowest responder rates during standard therapy. Thus due to its ability to induce type I T-cell responses in chronic HCV patients, the medicament according to the present invention is preferably used to replace or supplement ribavirin (i.e. IFN/IC41 instead IFN/riba).

The medicament according to the present invention can be also used in combination with small-molecule protease inhibitors. Preferably, it can be used for inducing type I T-cell responses in chronic HCV patients and/or for inducing similar T-cell responses as seen during/after successful standard therapy. The medicament according to the present invention hence can offer a mode-of-action distinctly different from small-molecule inhibitors. It can hence complement efficacy of small molecule inhibitors in terms of response rates, duration of response, relapse rates, optimal dose/schedule. The medicament according to the present invention has no significant side effects, can in particular improve the efficacy/side-effect ratio of a small molecule inhibitor.

The medicament according to the present invention can be used in combination with imiquimod (a TLR7 agonist in the already licensed product Aldara/3M). Especially the local mobilization of antigen-presenting cells can increase the immunological potency of the medicament according to the present invention. In particular, stronger responses against more peptides after less vaccinations can be expected; the immunological responder rates and duration of responses and hence also virological responses can be increased; the stronger T-cell responses against more peptides can overcome the RNA rebound effect e.g. as seen in the IC41-201 study using the medicament according to the present invention.

The present invention also encompasses a method of treating the HCV patients described herein with an effective amount of the described medicament.

The present invention is further illustrated by the following figures, examples from which further features, embodiments and advantages may be taken. It is to be understood that the present examples are given by way of illustration only and not by way of limitation of the disclosure.

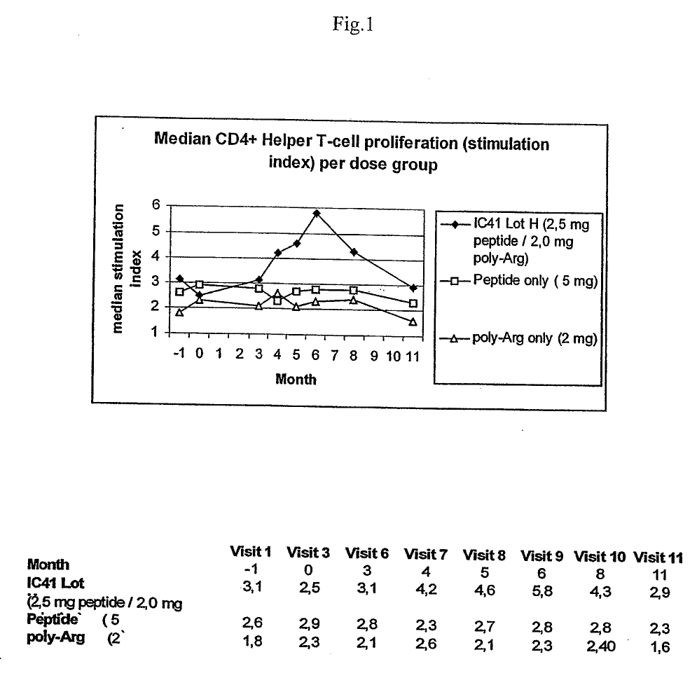

FIG. 1: CD4+ Helper-T-cell proliferation in chronic HCV patients. IC41 but not peptides alone, or poly-L-Arginine alone can induce significant proliferation. Vaccinations were given at month 0, 1, 2, 3, 4, 5. Stimulation indices for the IC41 peptides capable of inducing CD4+ Helper-T-cell responses were individually determined, significant responses were added up. Median responses of all 12 patients per dose group are shown.

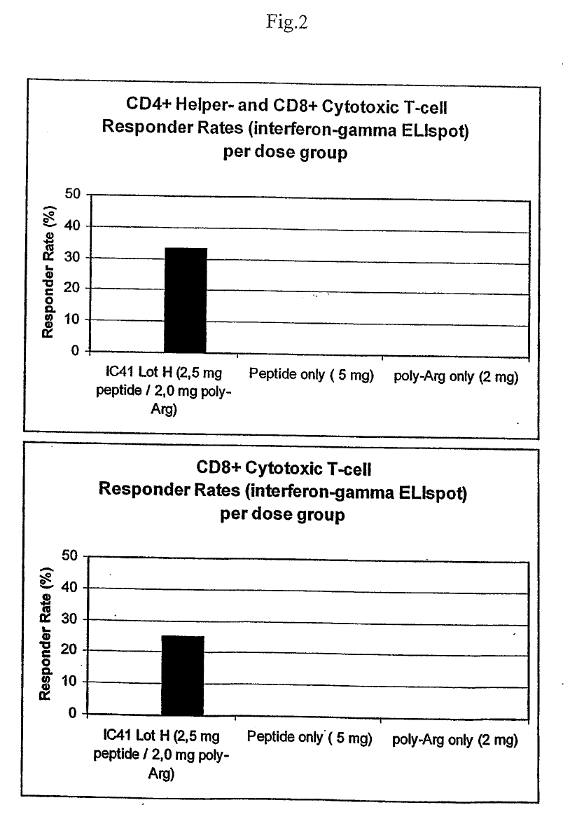

FIG. 2: Interferon-gamma ELIspot Responder Rates of patients vaccinated with IC41 or peptide alone or poly-L-Arginine alone. Each group consisted of 12 patients. Top panel: CD4+ Helper- and CD8+ cytotoxic T-cells, bottom panel: CD8+ cytotoxic T-cell, only.

### EXAMPLES

### Example I

### Human Clinical Study of HCV Peptide Vaccine in Chronic HCV Patients:

The first vaccine, where Poly-L-Arginine has been applied in humans is a fully synthetic therapeutic Hepatitis C Virus (HCV) vaccine. This vaccine was named IC41 and consists of a mixture of synthetic peptides representing conserved T cell epitopes of HCV plus Poly-L-Arginine as a synthetic T cell adjuvant. IC 41 comprises five peptides from different regions from the HCV polypeptide, i.a. the following three epitopes: HMWNFISGIQYLAGLSTLPGNPA (SEQ ID NO:11), CINGVCWTV (SEQ ID NO:54) and DLMGYIPAV (SEQ ID NO:51). The aim of this therapeutic approach is to restore a so-called type I T cell response against HCV in chronically infected patients. Such a response is typically seen in the around 15% of infected persons who do not proceed to chronicity but can clear HCV during the acute phase of infection. Since the pre-clinical experience with Poly-L-Arginine described earlier has shown its ability to induce type I immune responses in animal models it represents a promising T cell adjuvant for peptide vaccines for the treatment of HCV.

Various doses of the mentioned vaccine have already been tested in several clinical trials comprising more than 200 subjects: in an initial phase 1 study, safety and preliminary immunogenicity data of several doses were obtained. Results from that trial prompted initiation of a dose optimization study comprising 128 healthy volunteers in 10 different dose groups. The study was a randomized, single blind, parallel group, controlled study conducted to assess dose optimization and safety of the HCV peptide vaccine, IC41, in healthy subjects and was conducted in one center in Austria. One-hundred and twenty-eight subjects were randomly assigned to receive one of seven different doses and ratios of HCV peptide vaccine with Poly-L-Arginine, HCV peptide vaccine alone, Poly-L-Arginine alone or saline solution. All subjects received four administered vaccinations in monthly intervals. Immunogenicity was assessed at each of these time points at one month respectively and three months after the last vaccination.

The T cell stimulatory efficacy of Poly-L-Arginine was tested in a phase 2 clinical trial in chronic Hepatitis C Virus patients, who did not respond to or relapsed from standard interferon/ribavirin therapy.

For the present invention, a randomized, double blind study of HCV peptide vaccine, IC41, was conducted in patients with chronic HCV who had not responded to or had relapsed from primary standard HCV therapy. The study was conducted in 11 centers in Germany, Austria and Poland. Sixty patients were randomly assigned to receive three different doses and rations of HCV peptide vaccine with Poly-L-Arginine, HCV peptide vaccine alone or Poly-L-Arginine alone.

Male and female patients who met the following inclusion criteria were included in the study:
Diagnosis of chronic hepatitis C, with a documented course of at least six months (ALT>1.5 times the upper limit of normal [ULN] at two or more timepoints).
Non-response to or relapse from primary standard HCV therapy of six to 12 months (depending on the genotype of HCV).
HCV-RNA positive.
HLA-A2 positive.
HCV antibodies positive.
Liver biopsy within 30 months prior to inclusion, demonstrating hepatic inflammation and/or fibrosis.
Hematology and biochemistry laboratory results within the limits normally expected for the patient population (liver values maximum of 5×ULN).
Aged 18 to 65 years.
Written informed consent obtained prior to study entry.

In the present clinical study, each group consisted of 12 subjects, positive for HLA-A2. Subjects received 6 vaccinations in monthly intervals (at visits 3 to 8). Blood for immunological analyses was drawn prior vaccination and at visits 6 to 8, one month after last vaccination (visit 9), 3 months after last vaccination (visit 10) and 6 months after last vaccination (visit 11). For immunological monitoring of clinical trials, state-of-the-art T cell assays to determine immunological endpoints under GLP/GCP compliance were applied: Interferon-gamma ELIspot Assay, T cell Proliferation Assay, HLA-tetramer/FACS assay. These assays allow reliable measurements of epitope-specific T cell responses induced by the therapeutic HCV vaccine IC41. The vaccine-induced T cell immune responses serve as surrogate parameters of efficacy (Keilholz et al. 2002).

As primary endpoint T-cell immunogenicity was determined by a T-cell proliferation assay. The proliferation assay allows detection of peptide-specific T cells in biological samples like human blood. The basis of the assay is that, T cells upon stimulation with a peptide specifically recognized by their T cell receptor, react by secretion of cytokines and subsequent proliferation. Proliferation of cells can be measured by a variety of means; among the most sensitive approaches ranks incorporation of radioactively labeled thymidine into DNA synthesized prior cell division. This reaction can be carried out in a 96-well plate. Each well contains a fixed number of cells, which are cultured in the presence of antigen/peptide for a couple of days. For the last 16-20 hours thymidine labelled with tritium (3H-thymidine) is added. Cells are then harvested onto a filter plate: medium containing free radioactivity is washed away, whereas DNA sticks to the filter. Incorporated radioactivity can be quantified by means of a beta-scintillation counter determining counts-per-minute (cpm). The usual output is given as stimulation index (S.I.), which is defined as cpm of the test sample divided through cpm of the negative control.

As secondary endpoints T-cell immunogenicity was determined by interferon gamma ELIspot. ELIspot allows quantification of peptide-specific, functional (i.e. cytokine-secreting) T cells in biological samples like human blood. The basis of the assay is that, T cells upon stimulation with a peptide specifically recognized by the T cell receptor react by secretion of cytokines like IFN-γ. This reaction can be carried out in a 96-well plate.

The filter-wells of this plate are coated with a Mab specific for IFN-γ. Consequently, each cell secreting IFN-γ leaves an IFN-γ spot, which can be visualized with a subsequent color reaction. Spots can be counted using automated plate readers. Numbers obtained are a measure for the frequency of peptide-specific, IFN-γ-secreting T cells in the sample.

As additional secondary endpoint HLA-tetramer/FACS analysis was performed. HLA class I tetramers, soluble recombinant forms of a complex of HLA molecule and antigenic peptide, bind the antigen-specific T cell receptor used for T cell recognition. By using flow cytometry with fluorescent tetramers, antigen-specific CD8+ T lymphocytes can be reliably enumerated and characterized. The assay uses HLA-A*0201 custom-made iTag™-tetramers produced by Beckman Coulter Immunomics complexed with IC41 class I epitopes.

Subjects were classified as responders if they showed significant T-cell responses at any of visits 4 to 11 and had no response prior treatment. In the case of pre-existing immunity (significant T-cell response against any peptide within IC41 already prior vaccination), an increase of at least 3 times of the pre-existing value was required to classify the effect as response.

As a first important result, these clinical trials confirmed the excellent safety profile of completely synthetic peptides in general and Poly-L-Arginine in particular. Furthermore, several important lessons regarding activation of human T cells were learned: in both studies, T cell responses were assessed using [³H]-thymidine proliferation and IFN-γ ELIspot assays, and flow cytometry (FACS). These assays, which have been standardized and validated at Intercell AG's Clinical Immunology Laboratory enable reliable measurements of epitope-specific T cell responses induced by vaccination. All assays were performed in compliance with Good Laboratory Practice (GLP)/Good Clinical Practice (GCP) requirements. Standardization of the blood cell isolation procedure at the different investigational sites led to a high rate of evaluable assays. However, due to the lack of inter-laboratory standardization of T cell assays, comparison of the results of this study with published data from similar trials is difficult. Cryo-preserved blood cells were used, resulting in possible underestimation of T cell responses compared with assays that utilize fresh blood.

In the phase 2 study population of chronic HCV patients a slightly different picture was obtained: in general, CD4+ and CD8+ T cell responses to IC41 peptides were more frequent and more vigorous in peripheral blood samples from those patients who were immunized with peptide and Poly-L-Arginine together, than in samples from those patients who were immunized with peptide or Poly-L-Arginine alone, confirming the requirement of Poly-L-Arginine as a T cell adjuvant. Vaccine responder rates were approximately two to three-fold higher in the verum groups than in the control groups (FIG. 1). T cell proliferation responders were more numerous in the verum groups (30-60%) than in the control groups (0-17%).

Most importantly however, interferon-gamma ELIspot responders were observed exclusively in the verum groups (FIG. 2). These results demonstrated for the first time that Poly-L-Arginine is able to induce type I responses even in the setting of chronic HCV infection in patients who could not be cured by the interferon/ribavirin standard therapy. Significant Interferon-gamma ELIspot responses were detected against both HLA-class II (recognized by helper T cells) and HLA-class I (recognized by cytotoxic T cells) peptides. Analysis of data up to and including those obtained at the second immunological check (Visit 11) performed 6 months after the last immunization revealed no important differences in immunological responder rates compared with the data obtained at Visits 1 to 10, indicating sustainability of the IC41-induced immune response. The study also disclosed that T cell immunity against the virus can be raised to a level that is not too different from the one induced in healthy vaccines. Thus, immunosuppression may not be as prevalent as anticipated in patients. It remains to be elucidated, how such T cell responses can be optimally applied to reduce disease progression or ameliorate symptoms and eventually clear the infection.

Taken together, Poly-L-Arginine represents one of the first synthetic T cell adjuvants, which has consistently—from in vitro experiments up to incurable chronically infected patients—been able to induce and augment the desired kind of immune response. Its easy manufacturability, excellent safety profile and its efficacy even in such difficult settings as chronic HCV infection, make it a promising new tool in the fight against infectious diseases and cancer.

### Example II

### CD4+ Helper-T-Cell Proliferation in Chronic HCV Patients Can Be Induced by IC41, but not By Peptides Alone, or Poly-L-Arginine Alone

As shown in FIG. 1, IC41 is able of inducing a significant proliferative response in chronic HCV patients. Interestingly, neither peptides alone nor poly-L-Arginine alone have this ability, proofing the adjuvant effect of poly-L-Arginine.

### Example III

### Poly-L-Arginine is Required to Induce Type I (Interferon Gamma) T-Cell Responses in Chronic HCV Patients

As shown in FIG. 2, IC41 can induce interferon gamma secreting T-cells in chronic HCV patients, whereas peptides alone or poly-L-Arginine alone cannot induce any response. Importantly, both CD4+ Helper-T-cells and CD8+ cytotoxic T-cells can be induced.

Interferon gamma secretion is a hallmark of type I T-cell responses. Such responses are seen during the acute phase of infection in the subset of HCV patients, who eliminate the virus and do not proceed to chronic infection. Type I T-cell responses are also seen in patients undergoing standard therapy with interferon and ribavirin. Thus, induction of type I T-cell responses as achieved by IC41 is a primary goal of therapeutic vaccination against HCV.

### REFERENCES

Bellentani S, Miglioli L, Masutti F, Saccoccio G, Tiribelli C. Epidemiology of hepatitis C virus infection in Italy: the slowly unraveling mystery. Microbes Infect. 2000 November; 2(14):1757-63.

Liang T J, Rehermann B, Seeff L B, Hoofnagle J H. Pathogenesis, natural history, treatment, and prevention of hepatitis C. Ann Intern Med. 2000 Feb. 15; 132(4):296-305.

Cornberg M, Wedemeyer H, Manns M P. Treatment of chronic hepatitis C with PEGylated interferon and ribavirin. Curr Gastroenterol Rep. 2002 February; 4(1):23-30.

Keilholz U, Weber J, Finke J H, Gabrilovich D I, Kast W M, Disis M L, Kirkwood J M, Scheibenbogen C, Schlom J, Maino V C, Lyerly H K, Lee P P, Storkus W, Marincola F, Worobec A, Atkins M B.

Immunologic monitoring of cancer vaccine therapy: results of a workshop sponsored by the Society for Biological Therapy. J Immunother. 2002 March-April; 25(2):97-138. Review.

Fried M, Shiffman M, et al. Peginterferon Alfa-2a plus ribavirin for chronic hepatitis C virus infection. N Engl J Med, Vol. 347, No. 13. Sep. 26, 2002; 975-982.

1.-9. (canceled)

10. A method of treating a subject comprising:
obtaining a pharmaceutical composition comprising at least one polycationic compound and at least one HCV antigen or HCV epitope; and
administering the pharmaceutical composition to the subject.

11. The method of claim 10, wherein the subject is a human.

12. The method of claim 10, wherein the subject has a chronic HCV infection.

13. The method of claim 10, further defined as a method of vaccinating the subject.

14. The method of claim 10, wherein the subject has not responded to, partially responded to, or relapsed from primary standard HCV therapy by a combination of pegylated interferon-alpha and the antiviral ribavirin.

15. The method of claim 10, wherein the at least one polycationic compound is a polycationic peptide.

16. The method of claim 15, wherein the polycationic peptide contains between 20 and 500 amino acid residues.

17. The method of claim 16, wherein the polycationic peptide contains between 30 and 200 amino acid residues.

18. The method of claim 15, wherein the polycationic peptide is polyarginine or polylysine.

19. The method of claim 10, wherein CD4+ Helper-T-cells and CD8+ cytotoxic T-cells are induced in the subject.

20. The method of claim 10, further defined as a method of replacing or supplementing an HCV standard therapy with interferon alpha and ribavirin.

21. The method of claim 20, wherein the standard therapy is not effective or no longer effective.


SEQUENCE LISTING (115 sequences)

SEQ ID NO: 1 - protein, 36 aa, Artificial
  Synthetic peptide
RLAGLLRKGGEKIGEKLKKIGQKIKNFFQKLVPQPE

SEQ ID NO: 2 - protein, 9 aa, Artificial
  Synthetic peptide
XZXZZZXZX

SEQ ID NO: 3 - protein, 10 aa, Artificial
  Synthetic peptide
XZXZZZZXZX

SEQ ID NO: 4 - protein, 11 aa, Artificial
  Synthetic peptide
XZXZZZZZXZX

SEQ ID NO: 5 - protein, 12 aa, Artificial
  Synthetic peptide
XZXZZZZZZXZX

SEQ ID NO: 6 - protein, 13 aa, Artificial
  Synthetic peptide
XZXZZZZZZZXZX

SEQ ID NO: 7 - protein, 11 aa, Artificial
  Synthetic peptide
KLKLLLLLKLK

SEQ ID NO: 8 - protein, 20 aa, Artificial
  Synthetic peptide
MWNFISGIQYLAGLSTLPGN

SEQ ID NO: 9 - protein, 9 aa, Artificial
  Synthetic peptide
HMWNFISGI

SEQ ID NO: 10 - protein, 20 aa, Artificial
  Synthetic peptide
NFISGIQYLAGLSTLPGNPA

SEQ ID NO: 11 - protein, 23 aa, Artificial
  Synthetic peptide
HMWNFISGIQYLAGLSTLPGNPA

SEQ ID NO: 12 - protein, 26 aa, Artificial
  Synthetic peptide
IGLGKVLVDILAGYGAGVAGALVAFK

SEQ ID NO: 13 - protein, 16 aa, Artificial
  Synthetic peptide
AAWYELTPAETTVRLR

SEQ ID NO: 14 - protein, 20 aa, Artificial
  Synthetic peptide
DYPYRLWHYPCTVNYTIFKI

SEQ ID NO: 15 - protein, 20 aa, Artificial
  Synthetic peptide
DYPYRLWHYPCTVNFTIFKI

SEQ ID NO: 16 - protein, 10 aa, Artificial
  Synthetic peptide
AYSQQTRGLL

SEQ ID NO: 17 - protein, 12 aa, Artificial
  Synthetic peptide
TAYSQQTRGLLG

SEQ ID NO: 18 - protein, 13 aa, Artificial
  Synthetic peptide
SMSYTWTGALITP

SEQ ID NO: 19 - protein, 10 aa, Artificial
  Synthetic peptide
SPGALVVGVI

SEQ ID NO: 20 - protein, 8 aa, Artificial
  Synthetic peptide
LPRRGPRL

SEQ ID NO: 21 - protein, 15 aa, Artificial
  Synthetic peptide
GSIGLGKVLVDILAG

SEQ ID NO: 22 - protein, 15 aa, Artificial
  Synthetic peptide
IGLGKVLVDILAGYG

SEQ ID NO: 23 - protein, 15 aa, Artificial
  Synthetic peptide
LGKVLVDILAGYGAG

SEQ ID NO: 24 - protein, 15 aa, Artificial
  Synthetic peptide
LVDILAGYGAGVAGA

SEQ ID NO: 25 - protein, 15 aa, Artificial
  Synthetic peptide
DILAGYGAGVAGALV

SEQ ID NO: 26 - protein, 15 aa, Artificial
  Synthetic peptide
LAGYGAGVAGALVAF

SEQ ID NO: 27 - protein, 15 aa, Artificial
  Synthetic peptide
AGAAWYELTPAETTV

SEQ ID NO: 28 - protein, 15 aa, Artificial
  Synthetic peptide
AAWYELTPAETTVRL

SEQ ID NO: 29 - protein, 15 aa, Artificial
  Synthetic peptide
TAYSQQTRGLLGCIV

SEQ ID NO: 30 - protein, 18 aa, Artificial
  Synthetic peptide
VVCCSMSYTWTGALITPC

SEQ ID NO: 31 - protein, 32 aa, Artificial
  Synthetic peptide
VDYPYRLWHYPCTVNFTIFKVRMYVGGVEHRL

SEQ ID NO: 32 - protein, 15 aa, Artificial
  Synthetic peptide
DYPYRLWHYPCTVNF

SEQ ID NO: 33 - protein, 15 aa, Artificial
  Synthetic peptide
YPYRLWHYPCTVNFT

SEQ ID NO: 34 - protein, 15 aa, Artificial
  Synthetic peptide
LWHYPCTVNFTIFKV

SEQ ID NO: 35 - protein, 15 aa, Artificial
  Synthetic peptide
TVNFTIFKVRMYVGG

SEQ ID NO: 36 - protein, 15 aa, Artificial
  Synthetic peptide
TIFKVRMYVGGVEHR

SEQ ID NO: 37 - protein, 32 aa, Artificial
  Synthetic peptide
VDYPYRLWHYPCTVNYTIFKIRMYVGGVEHRL

SEQ ID NO: 38 - protein, 15 aa, Artificial
  Synthetic peptide
DYPYRLWHYPCTVNY

SEQ ID NO: 39 - protein, 15 aa, Artificial
  Synthetic peptide
LWHYPCTVNYTIFKI

SEQ ID NO: 40 - protein, 15 aa, Artificial
  Synthetic peptide
TVNYTIFKIRMYVGG

SEQ ID NO: 41 - protein, 15 aa, Artificial
  Synthetic peptide
TIFKIRMYVGGVEHR

SEQ ID NO: 42 - protein, 11 aa, Artificial
  Synthetic peptide
RMYVGGVEHRL

SEQ ID NO: 43 - protein, 29 aa, Artificial
  Synthetic peptide
KFPGGGQIVGGVYLLPRRGPRLGVRATRK

SEQ ID NO: 44 - protein, 22 aa, Artificial
  Synthetic peptide
KFPGGGQIVGGVYLLPRRGPRL

SEQ ID NO: 45 - protein, 10 aa, Artificial
  Synthetic peptide
YLLPRRGPRL

SEQ ID NO: 46 - protein, 9 aa, Artificial
  Synthetic peptide
GPRLGVRAT

SEQ ID NO: 47 - protein, 9 aa, Artificial
  Synthetic peptide
RLGVRATRK

SEQ ID NO: 48 - protein, 14 aa, Artificial
  Synthetic peptide
GYKVLVLNPSVAAT

SEQ ID NO: 49 - protein, 10 aa, Artificial
  Synthetic peptide
AYAAQGYKVL

SEQ ID NO: 50 - protein, 19 aa, Artificial
  Synthetic peptide
AYAAQGYKVLVLNPSVAAT

SEQ ID NO: 51 - protein, 9 aa, Artificial
  Synthetic peptide
DLMGYIPAV

SEQ ID NO: 52 - protein, 10 aa, Artificial
  Synthetic peptide
GYIPLVGAPL

SEQ ID NO: 53 - protein, 13 aa, Artificial
  Synthetic peptide
DLMGYIPLVGAPL

SEQ ID NO: 54 - protein, 9 aa, Artificial
  Synthetic peptide
CINGVCWTV

SEQ ID NO: 55 - protein, 16 aa, Artificial
  Synthetic peptide
GEVQVVSTATQSFLAT

SEQ ID NO: 56 - protein, 25 aa, Artificial
  Synthetic peptide
GEVQVVSTATQSFLATCINGVCWTV

SEQ ID NO: 57 - protein, 9 aa, Artificial
  Synthetic peptide
QYLAGLSTL

SEQ ID NO: 58 - protein, 20 aa, Artificial
  Synthetic peptide
DYPYRLWHYPCTVNFTIFKV

SEQ ID NO: 59 - protein, 9 aa, Artificial
  Synthetic peptide
DYPYRLWHY

SEQ ID NO: 60 - protein, 9 aa, Artificial
  Synthetic peptide
TVNYTIFKI

SEQ ID NO: 61 - protein, 8 aa, Artificial
  Synthetic peptide
TINYTIFK

SEQ ID NO: 62 - protein, 9 aa, Artificial
  Synthetic peptide
TVNFTIFKV

SEQ ID NO: 63 - protein, 10 aa, Artificial
  Synthetic peptide
HYPCTVNYTI

SEQ ID NO: 64 - protein, 10 aa, Artificial
  Synthetic peptide
HYPCTVNFTI

SEQ ID NO: 65 - protein, 10 aa, Artificial
  Synthetic peptide
RMYVGGVEHR

SEQ ID NO: 66 - protein, 9 aa, Artificial
  Synthetic peptide
TPAETTVRL

SEQ ID NO: 67 - protein, 23 aa, Artificial
  Synthetic peptide
GWRLLAPITAYSQQTRGLLGCIV

SEQ ID NO: 68 - protein, 15 aa, Artificial
  Synthetic peptide
GQGWRLLAPITAYSQ

SEQ ID NO: 69 - protein, 9 aa, Artificial
  Synthetic peptide
RLLAPITAY

SEQ ID NO: 70 - protein, 25 aa, Artificial
  Synthetic peptide
GQGWRLLAPITAYSQQTRGLLGCIV

SEQ ID NO: 71 - protein, 22 aa, Artificial
  Synthetic peptide
GQGWRLLAPITAYSQQTRGLLG

SEQ ID NO: 72 - protein, 9 aa, Artificial
  Synthetic peptide
AYSQQTRGL

SEQ ID NO: 73 - protein, 9 aa, Artificial
  Synthetic peptide
ILAGYGAGV

SEQ ID NO: 74 - protein, 9 aa, Artificial
  Synthetic peptide
VAGALVAFK

SEQ ID NO: 75 - protein, 10 aa, Artificial
  Synthetic peptide
GYGAGVAGAL

SEQ ID NO: 76 - protein, 10 aa, Artificial
  Synthetic peptide
SMSYTWTGAL

SEQ ID NO: 77 - protein, 9 aa, Artificial
  Synthetic peptide
SYTWTGALI

SEQ ID NO: 78 - protein, 16 aa, Artificial
  Synthetic peptide
FTDNSSPPAVPQTFQV

SEQ ID NO: 79 - protein, 19 aa, Artificial
  Synthetic peptide
LEDRDRSELSPLLLSTTEW

SEQ ID NO: 80 - protein, 16 aa, Artificial
  Synthetic peptide
LEDRDRSELSPLLLST

SEQ ID NO: 81 - protein, 9 aa, Artificial
  Synthetic peptide
RSELSPLLL

SEQ ID NO: 82 - protein, 9 aa, Artificial
  Synthetic peptide
ELSPLLLST

SEQ ID NO: 83 - protein, 10 aa, Artificial
  Synthetic peptide
DRDRSELSPL

SEQ ID NO: 84 - protein, 9 aa, Artificial
  Synthetic peptide
LEDRDRSEL

SEQ ID NO: 85 - protein, 9 aa, Artificial
  Synthetic peptide
LEDRDRSEL

SEQ ID NO: 86 - protein, 21 aa, Artificial
  Synthetic peptide
YLVAYQATVCARAQAPPPSWD

SEQ ID NO: 87 - protein, 9 aa, Artificial
  Synthetic peptide
YLVAYQATV

SEQ ID NO: 88 - protein, 17 aa, Artificial
  Synthetic peptide
MSTNPKPQRKTKRNTNR

SEQ ID NO: 89 - protein, 11 aa, Artificial
  Synthetic peptide
PQRKTKRNTNR

SEQ ID NO: 90 - protein, 9 aa, Artificial
  Synthetic peptide
QRKTKRNTN

SEQ ID NO: 91 - protein, 9 aa, Artificial
  Synthetic peptide
RKTKRNTNR

SEQ ID NO: 92 - protein, 9 aa, Artificial
  Synthetic peptide
MSTNPKPQR

SEQ ID NO: 93 - protein, 9 aa, Artificial
  Synthetic peptide
MSTNPKPQK

SEQ ID NO: 94 - protein, 21 aa, Artificial
  Synthetic peptide
LINTNGSWHINRTALNCNDSL

SEQ ID NO: 95 - protein, 17 aa, Artificial
  Synthetic peptide
NGSWHINRTALNCNDSL

SEQ ID NO: 96 - protein, 10 aa, Artificial
  Synthetic peptide
LINTNGSWHI

SEQ ID NO: 97 - protein, 10 aa, Artificial
  Synthetic peptide
RTALNCNDSL

SEQ ID NO: 98 - protein, 16 aa, Artificial
  Synthetic peptide
LINTNGSWHINRTALN

SEQ ID NO: 99 - protein, 10 aa, Artificial
  Synthetic peptide
SWHINRTALN

SEQ ID NO: 100 - protein, 15 aa, Artificial
  Synthetic peptide
TTILGIGTVLDQAET

SEQ ID NO: 101 - protein, 9 aa, Artificial
  Synthetic peptide
TTILGIGTV

SEQ ID NO: 102 - protein, 9 aa, Artificial
  Synthetic peptide
TILGIGTVL

SEQ ID NO: 103 - protein, 18 aa, Artificial
  Synthetic peptide
FDSSVLCECYDAGAAWYE

SEQ ID NO: 104 - protein, 15 aa, Artificial
  Synthetic peptide
FDSSVLCECYDAGCA

SEQ ID NO: 105 - protein, 10 aa, Artificial
  Synthetic peptide
VLCECYDAGA

SEQ ID NO: 106 - protein, 15 aa, Artificial
  Synthetic peptide
VVLCECYDAGAAWYE

SEQ ID NO: 107 - protein, 20 aa, Artificial
  Synthetic peptide
ARLIVFPDLGVRVCEKMALY

SEQ ID NO: 108 - protein, 9 aa, Artificial
  Synthetic peptide
ARLIVFPDL

SEQ ID NO: 109 - protein, 10 aa, Artificial
  Synthetic peptide
RLIVFPDLGV

SEQ ID NO: 110 - protein, 9 aa, Artificial
  Synthetic peptide
RVCEKMALY

SEQ ID NO: 111 - protein, 15 aa, Artificial
  Synthetic peptide
AFCSAMYVGDLCGSV

SEQ ID NO: 112 - protein, 15 aa, Artificial
  Synthetic peptide
GVLFGLAYFSMVGNW

SEQ ID NO: 113 - protein, 15 aa, Artificial
  Synthetic peptide
TRVPYFVRAQGLIRA

SEQ ID NO: 114 - protein, 15 aa, Artificial
  Synthetic peptide
TTLLFNILGGWVAAQ

SEQ ID NO: 115 - protein, 10 aa, Artificial
  Synthetic peptide
LLFNILGGWV